(19) United States
(12) Patent Application Publication
(10) Pub. No.: US 2015/0216667 A1
(43) Pub. Date: Aug. 6, 2015

(54) TAPERED ADAPTER

(71) Applicant: Zimmer, Inc., Warsaw, IN (US)

(72) Inventor: Matthew E. Monaghan, Fort Wayne, IN (US)

(21) Appl. No.: 14/590,356

(22) Filed: Jan. 6, 2015

Related U.S. Application Data
(60) Provisional application No. 61/935,418, filed on Feb. 4, 2014.

(51) Int. Cl. A61F 2/30

(52) U.S. Cl. CPC A61F 2/30; A61F 2002/30062; A61F 2002/30324; A61F 2002/30604; A61F 2002/30354

(57) ABSTRACT

A prosthesis including an adapter and associated methods are provided herein. The prosthesis includes a stem including a stem outer surface, a distal portion, and a proximal portion. An adapter includes an adapter outer surface and a bore including an adapter inner surface, wherein the stem outer surface is substantially congruent with the adapter inner surface such that the proximal portion of the stem is configured to be at least partially received by the bore.

### CLAIM OF PRIORITY

This application claims the benefit of U.S. Provisional Patent Application Ser. No. 61/935,418, filed on Feb. 4, 2014, the benefit of priority of which is claimed hereby, and which is incorporated by reference herein in its entirety.

### BACKGROUND

Prosthetic devices, such as prosthetic implants, can replace or augment body components or portions of body components that cannot be regenerated or are no longer functioning properly. Examples of prosthetic implants include heart valves, spinal implants, dental implants, collagen for soft tissue augmentation, and orthopedic devices, such as artificial knee, hip, and ankle joints.

Some prosthetic implants can include components that articulate to mimic motion of a body component. For example, a humeral or a femoral ball joint in combination with a stem component can be used in hip or shoulder arthroplasty procedures. Such stem components can be subjected to large amounts of force or stress during normal use.

### SUMMARY

The present inventor has recognized, among other things, that a larger taper on a stem for an orthopedic implant can increase the load-bearing capability, fatigue strength or corrosion resistance of the stem. However, producing a new stem configured to couple to existing head components can be cost prohibitive.

One way to increase the taper on existing stem components can be providing an adapter configured to be retrofit to an existing stem of a prosthetic implant to improve strength of the prosthetic implant, such as a load or a stress bearing capability.

To better illustrate the adapter and related methods disclosed herein, a non-limiting list of examples is provided here:

In Example 1, a prosthesis includes a stem including a stem outer surface, a distal portion, and a proximal portion; and an adapter including an adapter outer surface and a bore including an adapter inner surface, wherein the stem outer surface is substantially congruent with the adapter inner surface such that the proximal portion of the stem is configured to be at least partially received by the bore.

In Example 2, the prosthesis of Example 1 is optionally configured such that the stem outer surface defines a stem taper at the proximal portion, the adapter inner surface defining an adapter inner surface taper substantially congruent with the stem taper, and the adapter outer surface defines an adapter outer surface taper.

In Example 3, the prosthesis of any one or any combination of Examples 1-2 is optionally configured such that the stem taper is selected from the group consisting of a Brown and Sharpe taper, a Jacobs taper, a Jarno taper, a Morse taper, a National Machine Tool Builders Association (NMTB) taper, and an R8 taper.

In Example 4, the prosthesis of any one or any combination of Examples 1-3 is optionally configured such that the stem is non-removably coupled to the adapter.

In Example 5, the prosthesis of any one or any combination of Examples 1-4 is optionally configured such that the stem is coupled to the adapter by a biocompatible weld.

In Example 6, the prosthesis of any one or any combination of Examples 1-5 is optionally configured to further include a head including a receiving cavity configured to at least partially receive a proximal portion of the adapter.

In Example 7, the prosthesis of any one or any combination of Examples 1-6 is optionally configured such that the head is about 48 millimeters to about 70 millimeters in diameter.

In Example 8, the prosthesis of any one or any combination of Examples 1-7 is optionally configured such that the stem proximal portion includes a stem proximal end with a first dimension and the adapter includes an adapter proximal end with a second dimension equal to or greater than the first dimension.

In Example 9, the prosthesis of any one or any combination of Examples 1-8 is optionally configured such that the adapter outer surface taper is larger than the stem taper.

In Example 10, the prosthesis of any one or any combination of Examples 1-9 is optionally configured such that the adapter is configured to handle greater loads, better resistance to the torques transmitted by larger diameter heads to the stem, better fatigue strength and resistance to corrosion.

In Example 11, the prosthesis of any one or any combination of Examples 1-10 is optionally configured such that the adapter outer surface taper is selected from the group consisting of a Brown and Sharpe taper, a Jacobs taper, a Jarno taper, a

Morse taper, a National Machine Tool Builders Association (NMTB) taper, and an R8 taper.

In Example 12, a method for modifying a prosthesis stem includes providing or obtaining a stem including a stem outer surface, a distal portion, and a proximal portion, the stem outer surface defining a stem taper; and increasing a diameter of the proximal portion of the stem with an adapter including a bore with an adapter inner surface defining an adapter inner surface taper configured to contact the stem taper of the stem outer surface.

In Example 13, the method of Example 12 is optionally configured such that increasing the diameter includes non-removably coupling the adapter to the stem, the adapter including an adapter outer surface defining an adapter outer surface taper.

In Example 14, the method of any one or any combination Examples 12-13 is optionally configured such that the non-removably coupling includes welding the stem and the adapter.

In Example 15, the method of any one or any combination Examples 12-14 is optionally configured such that increasing the diameter includes increasing the load bearing capability of the stem.

In Example 16, the method of any one or any combination Examples 12-15 is optionally configured such to furthering include inserting a proximal end of the adapter at least partially into a receiving cavity of a head prosthesis

In Example 17, the method of any one or any combination Examples 12-16 is optionally configured such that providing or obtaining includes providing or obtaining the stem from a pre-existing inventory of stem components

In Example 18, the method of any one or any combination Examples 12-17 is optionally configured such that the adapter is configured to be coupled to a head with a first diameter and the stem is configured to be coupled a head with a second diameter less than the first diameter.

In Example 19, a prosthesis includes a stem including a stem outer surface, a distal portion, and a proximal portion, the stem outer surface defining a stem taper; and an adapter configured to be coupled to the proximal portion of the stem including an adapter outer surface defining an adapter outer surface taper and a bore with an adapter inner surface defining an adapter inner surface taper substantially congruent with the stem taper, wherein the stem proximal portion includes a stem proximal end with a first dimension and the adapter includes an adapter proximal end with a second dimension, wherein the second dimension is greater than or equal to the first dimension. In Example 20, the prosthesis of Example 19 is optionally configured such that the adapter outer surface is about 0.5 cm to about 2.0 cm in length.

In Example 21, the prosthesis or method of any one or any combination of Examples 1-20 is optionally configured such that all elements or options recited are available to use or select from.

These and other examples and features of the present prosthetic implants and methods will be set forth in part in the following Detailed Description. This Summary is intended to provide non-limiting examples of the present subject Matter—it is not intended to provide an exclusive or exhaustive explanation. The Detailed Description below is included to provide further information about the present tapered adapter and methods.

### BRIEF DESCRIPTION OF THE DRAWINGS

In the drawings, which are not necessarily drawn to scale, like numerals may describe similar components in different views. Letters may represent distances or dimensions. The drawings illustrate generally, by way of example, but not by way of limitation, various embodiments discussed in the present document.

FIG. 1 illustrates a cross-sectional view of an exemplary prosthesis including an adapter in accordance with at least one example of the present disclosure;

FIG. 2 illustrates a perspective view of an exemplary stem in accordance with at least one example of the present disclosure;

FIG. 3 illustrates a perspective view of an exemplary adapter in accordance with at least one example of the present disclosure; and

FIG. 4 illustrates a method for modifying a prosthesis stem in accordance with at least one example of the present disclosure.

### DETAILED DESCRIPTION

The present disclosure relates generally to an adapter configured to couple to a stem, so as to increase load-bearing capability of the stem. Orthopedic surgeons are typically interested in reducing the risk of dislocation of an implant, such as a hip, a shoulder, or a knee implant. Dislocation of the implant can result from abnormal patient anatomy, lax soft tissue, or other patient specific factors. In an example, one remedy for reducing the risk of dislocation can be to implant a hip prosthesis having a large diameter bearing interface, which increases the distance of separation between pelvic and femoral elements of the implant. In such an example, a large diameter spherical head and mating cup can be used to mate with a small diameter stem to maximize the range of motion of the joint. Because of the geometry of the small diameter stem, a connection interface between the stem and the head, such as a Morse taper, is also relatively reduced. The combination of the large diameter head and the small connection interface can result in a maximized load on the connection interface, leading to potential adverse biologic consequences, such as loss of the joint, pain, lack of motion, or revision resulting from, for example, metal debris, corrosion, or corrosion byproducts. The present inventor has recognized that by coupling an adapter to the smaller diameter stem, the present invention, can increase the connection interface, and consequently increase the load-bearing capability, fatigue strength or corrosion resistance of the stem or prosthesis.

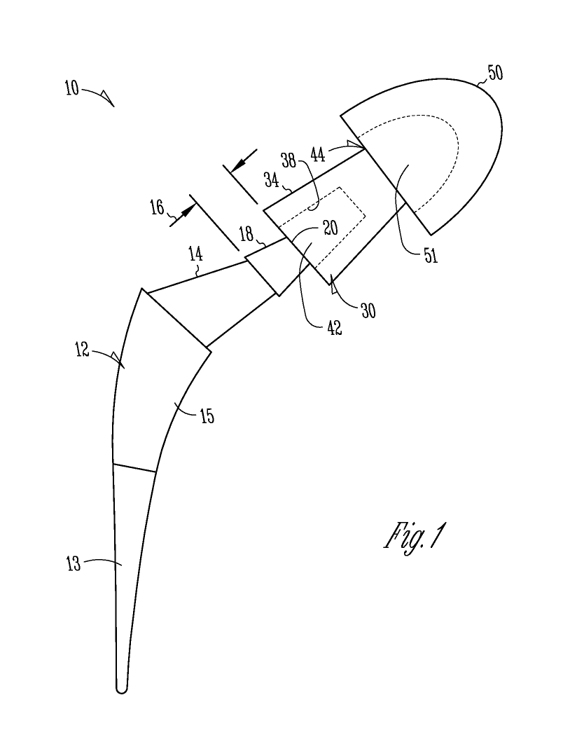

As shown in FIG. 1, an orthopedic prosthesis, such as a hip implant 10, can include a stem 12, an adapter 30, and a head 50. Although the orthopedic prosthesis in FIG. 1 is illustrated as the hip implant 10, other prosthetics are contemplated, such as any medical device suitable to replace a missing anatomical structure, support a damaged anatomical structure, or enhance an existing anatomical structure. Suitable exemplary implants include, but are not limited to, the components that constitute a reverse shoulder implant, a knee implant, an ankle implant, spinal implants, dental implants, an elbow implant, and the like. An implant material can include any biocompatible material suitable for implantation in a patient for any given length of time. Suitable biocompatible materials can include, for example, a metallic material such as at least one of a variety of stainless steel composites, titanium, chromium-cobalt, tantalum, or the like, or a non-metallic biocompatible material such as a biocompatible polymer or other plastic material including polyamide, polyphenylsulfone, polyethersulfone, polysulfone, polyketone, polyarylamide, polyether ether ketone (PEEK), polycarbonate, polystyrene, acrylonitrile butadiene styrene (ABS), acrylics, polyetherimide, polyimide, polyphenylsulfone, polymethoylmethacrylate, fiber filled variations of these polymers, amorphous polymeric material, or various other biocompatible polymers.

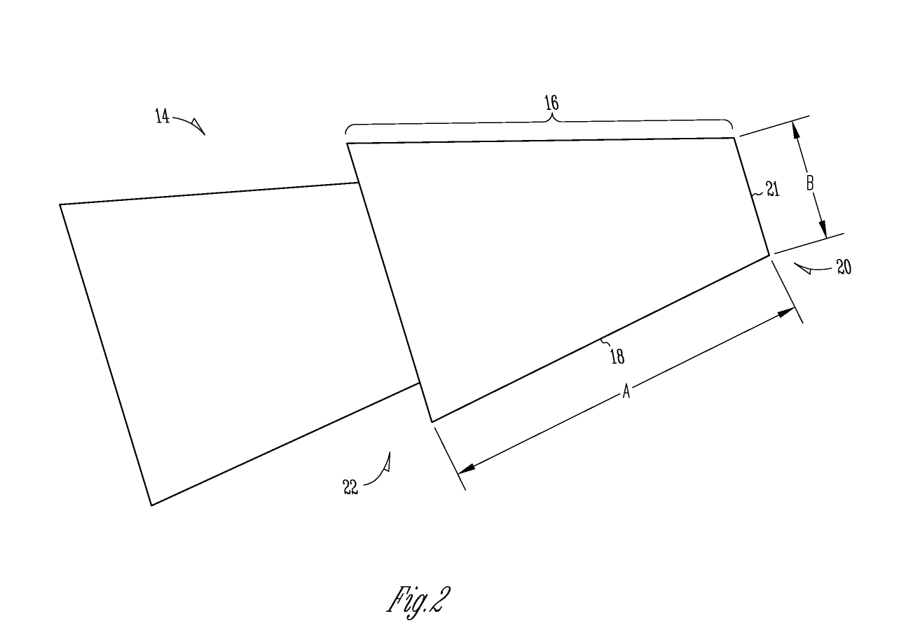

The stem 12 can include a neck portion 14 and a shank portion 13. The shank portion 13 can include a porous region 15, so as to permit bone or tissue ingrowth, configured to be received by a long bone of a patient. The porous region 15 can include a plurality of interconnecting interstitial cells configured such that bone or biological tissue ingrowth can occur within the cells. Although shown as a discrete and continuous region of the hip implant 10, the porous region 15 is not so limited. For example, the porous region 15 can include an outer surface of an implant, an interior surface of an implant, or any region of an implant that bone or biological tissue ingrowth is desired. The neck portion 14 can include a stem taper 16, as discussed herein in connection with FIG. 2, configured to be received by a bore 42 of the stem adapter 30.

The hip implant 10 can include a head 50 configured to engage a hemispherical shell, so as to mimic a natural hip. The head 50 can include a receiving cavity 51 configured to at least partially receive a proximal portion 44 of the adapter 30, such as an adapter outer surface 34. The stem 12, the adapter 30, and the head 50 can be configured to provide a pre-determined range of motion, so as to mimic the range of motion of a natural hip joint. In an example, the hip implant 10 can include the stem 12, the adapter 30, and the head 50 with a range of motion substantially the same as hip implant 10 without the adapter 30. That is, the addition of the adapter 50 can, in an example, be configured so as to not inhibit the range of motion of the hip implant 10. The adapter 30 can be configured to mate with a head 50 of any size, such as about 48 millimeters to about 70 millimeters in diameter.

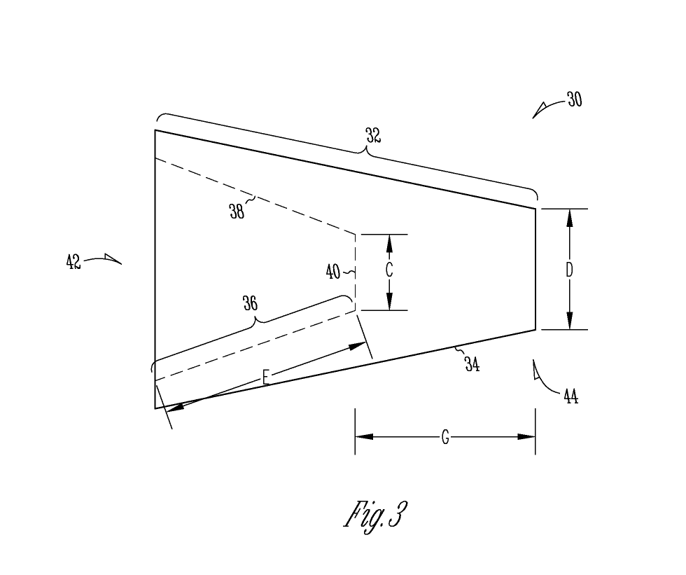

The adapter 30 can be configured to non-removably couple to the stem 12, such as by a biocompatible weld, sealing, adhesive, or a combination thereof. Welds can include, but are not limited to, arc welding, gas welding, resistance welding, energy beam welding, or solid-state welding. The proximal portion 20 of the stem 12 can be at least partially received by the bore 42, so as to provide a connection interface suitable for coupling the stem 12 with the adapter 30. For example, a stem outer surface 18 can be substantially congruent with an adapter inner surface 38 defined by the bore 42, as described herein in connection with FIGS. 2 and 3.

FIG. 2 illustrates a detailed side view of the neck portion 14 of the stem 12. The neck portion 14 can include the stem outer surface 18, the proximal portion 20, and a distal portion 22. The neck portion 14 can be formed from a biocompatible material, as described herein. The proximal portion 20 can be configured to be at least partially received by the adapter 30. For example, a proximal end 21 of the proximal portion 20 can define a length B sized and configured to be received by the bore 42. The stem outer surface 18 can have a length A, typically between about 0.5 cm and about 2.0 cm. The stem outer surface 18 can define a stem taper 16, such as a Brown and Sharpe taper, a Jacobs taper, a Jarno taper, a Morse taper, a National Machine Tool Builders Association (NMTB) taper, or an R8 taper. In an example, the stem taper 16 can be substantially the length A or a portion of the length A, such as about 0.1 A, 0.2 A, 0.3 A, 0.4 A, 0.5 A, 0.6 A, 0.7 A, 0.8 A, 0.95 A, or greater. The stem taper 16 can include tapers, such as six-degree, 12/14, or 16/18 tapers commonly used in hip implants. In an example, the stem 12 can be a pre-existing stem 12 manufactured to be mated with a pre-existing head, such as head 50.

FIG. 3 illustrates a cross-sectional view of the adapter 30. The adapter 30 can include the bore 42 having the adapter inner surface 38, so as to define an adapter inner surface taper 36. The adapter inner surface taper 36 can be substantially congruent to the stem taper 16, such that the length B is substantially the same as a length C of the bore 42. For example, the proximal end 21 can at least partially contact a bore abutment surface 40 when the stem 12 is engaged with the adapter 30. The adapter inner surface taper 36 can include, for example, a Brown and Sharpe taper, a Jacobs taper, a Jarno taper, a Morse taper, a National Machine Tool Builders Association (NMTB) taper, or an R8 taper. In an example, a length of the adapter inner surface E can be less than, substantially the same as, or greater than the length A of the stem outer surface 18. The bore 42, the adapter inner surface 38, the adapter inner surface taper 36, the bore abutment surface 40, or a combination thereof, can be configured to provide a sufficient contact interface with the stem taper 16, the proximal end 21, the proximal portion 20, the stem outer surface 18, or a combination thereof, so as to permit non-removably coupling the stem 12 with the adapter 30, such as by one or more of the securing means described herein.

The adapter 30 can be configured to be non-removably coupled to a stem having a smaller taper, such as the stem 12, so as to increase the load-handling capability of the stem 12. For example, the adapter outer surface taper 32 defined by the adapter outer surface 34 can have a larger taper than the proximal portion 20 of the stem 12 so as to provide a larger taper to couple to the head 50. With dimension 32 in FIG. 3 in typical ranges from 0.5 cm to 2.0 cm and dimension D in FIG. 3 typically from about 0.5 cm to about 1.0 cm. Surface C in FIG. 3 is typically less than about 1.0 cm in diameter. As discussed herein, the larger taper provided by the adapter outer surface taper 32 can provide increased load-handling capability at a connection between the stem 12 and the head 50. An overall length of length of the adapter E+G can be configured such that it provides a larger taper than the stem taper 16. Further, an adapter proximal end 44 can include a second dimension D equal to or greater than the first dimension B of the stem proximal end 21. The larger adapter outer surface taper 32 can be configured to couple to a larger head 50, as compared to the stem taper 16.

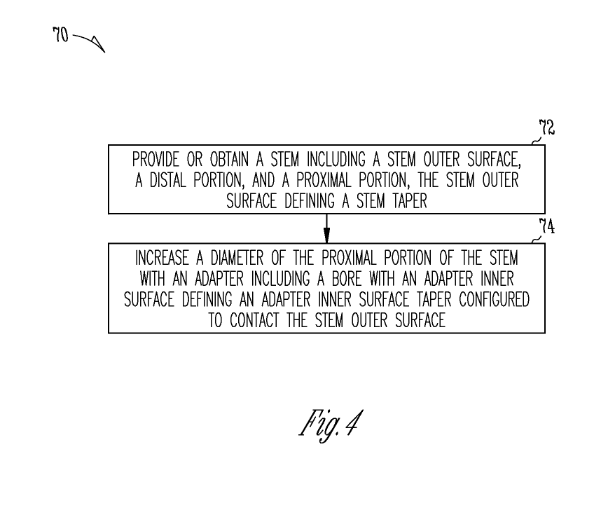

As illustrated in FIG. 4, a method 70 for modifying a prosthesis stem can include providing or obtaining a stem including a stem outer surface, a distal portion, and a proximal portion, the stem outer surface defining a stem taper 72.

The prosthesis stem can include stem 12 as described in connection with FIGS. 1-3 herein. The stem can be provided or obtained from a pre-existing stock or warehouse of stems, such that the stem can be retrofit as described herein.

The method 70 can include increasing a diameter of a proximal portion of the stem, such as a proximal end 21, with an adapter including a bore 42 with an adapter inner surface defining an adapter inner surface taper configured to contact an outer surface of the stem. Increasing the diameter can include non-removably coupling the adapter to the stem, the adapter including an adapter outer surface defining an adapter outer surface taper, such as a taper larger than the stem taper. Non-removably coupling can include securing the stem to the adapter, such as by biocompatible welding, sealing, adhering, or a combination thereof. In an example, increasing the diameter of the proximal end of the stem can include increasing the load bearing capability of the stem or the stem taper. Increasing the diameter can include increasing from the diameter B to the diameter D, as discussed herein.

The above Detailed Description includes references to the accompanying drawings, which form a part of the Detailed Description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects. As used herein, the term “about” when used in connection with a range or a value of dimension indicate within machine or manufacturing tolerance ranges.

The above Detailed Description is intended to be illustrative, and not restrictive. For example, the above-described examples (or one or more aspects thereof) may be used in combination with each other. Other embodiments can be used, such as by one of ordinary skill in the art upon reviewing the above Detailed Description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

What is claimed is:

1. A prosthesis, comprising:
a stem including a stem outer surface, a distal portion, and a proximal portion; and
an adapter including an adapter outer surface and a bore including an adapter inner surface, wherein the stem outer surface is substantially congruent with the adapter inner surface such that the proximal portion of the stem is configured to be at least partially received by the bore.

2. The prosthesis of claim 1, wherein the stem outer surface defines a stem taper at the proximal portion, the adapter inner surface defining an adapter inner surface taper substantially congruent with the stem taper, and the adapter outer surface defines an adapter outer surface taper.

3. The prosthesis of claim 2, wherein the stem taper is selected from the group consisting of a Brown and Sharpe taper, a Jacobs taper, a Jarno taper, a Morse taper, a National Machine Tool Builders Association (NMTB) taper, and an R8 taper.

4. The prosthesis of claim 1, wherein the stem is non-removably coupled to the adapter.

5. The prosthesis of claim 4, wherein the stem is coupled to the adapter by a biocompatible weld.

6. The prosthesis of claim 1, further comprising a head including a receiving cavity configured to at least partially receive a proximal portion of the adapter.

7. The prosthesis of claim 1, wherein the head is about 48 millimeters to about 70 millimeters in diameter.

8. The prosthesis of claim 1, wherein the stem proximal portion includes a stem proximal end with a first dimension and the adapter includes an adapter proximal end with a second dimension equal to or greater than the first dimension.

9. The prosthesis of claim 2, wherein the adapter outer surface taper is larger than the stem taper.

10. The prosthesis of claim 1, wherein the adapter is configured to handle greater loads, better resistance to the torques transmitted by larger diameter heads to the stem, better fatigue strength and resistance to corrosion.

11. The prosthesis of claim 2, wherein the adapter outer surface taper is selected from the group consisting of a Brown and Sharpe taper, a Jacobs taper, a Jarno taper, a Morse taper, a National Machine Tool Builders Association (NMTB) taper, and an R8 taper.

12. A method for modifying a prosthesis stem, comprising:
providing or obtaining a stem including a stem outer surface, a distal portion, and a proximal portion, the stem outer surface defining a stem taper; and
increasing a diameter of the proximal portion of the stem with an adapter including a bore with an adapter inner surface defining an adapter inner surface taper configured to contact the stem taper of the stem outer surface.

13. The method of claim 12, wherein increasing the diameter includes non-removably coupling the adapter to the stem, the adapter including an adapter outer surface defining an adapter outer surface taper.

14. The method of claim 13, wherein non-removably coupling includes welding the stem and the adapter.

15. The method of claim 12, wherein increasing the diameter includes increasing the load bearing capability of the stem.

16. The method of claim 12, furthering comprising inserting a proximal end of the adapter at least partially into a receiving cavity of a head prosthesis.

17. The method of claim 12, wherein providing or obtaining includes providing or obtaining the stem from a pre-existing inventory of stem components.

18. The method of claim 12, wherein the adapter is configured to be coupled to a head with a first diameter and the stem is configured to be coupled a head with a second diameter less than the first diameter.

19. A prosthesis, comprising:
a stem including a stem outer surface, a distal portion, and a proximal portion, the stem outer surface defining a stem taper; and
an adapter configured to be coupled to the proximal portion of the stem including an adapter outer surface defining an adapter outer surface taper and a bore with an adapter inner surface defining an adapter inner surface taper substantially congruent with the stem taper,
wherein the stem proximal portion includes a stem proximal end with a first dimension and the adapter includes an adapter proximal end with a second dimension, wherein the second dimension is greater than or equal to the first dimension.

20. The prosthesis of claim 19, wherein the adapter outer surface is about 0.5 cm to about 2.0 cm in length.
